(19) United States
(12) Patent Application Publication
(10) Pub. No.: US 2023/0407269 A1
(43) Pub. Date: Dec. 21, 2023

(54) Vaccine

(71) Applicant: Erez Yahalomi, Yehud Monosson (IL)

(72) Inventor: Erez Yahalomi, Yehud Monosson (IL)

(21) Appl. No.: 18/033,809

(22) PCT Filed: Oct. 26, 2021
(86) PCT No.: PCT/IL2021/051264

§ 371 Date: Apr. 26, 2023

Related U.S. Application Data
(60) Provisional application No. 63/105,925, filed on Oct. 27, 2020.
(60) Provisional application No. 63/142,289, filed on Jan. 27, 2021.
(60) Provisional application No. 63/149,368, filed on Feb. 15, 2021.
(60) Provisional application No. 63/193,623, filed on May 27, 2021.

(51) Int. Cl. C12N 7/00; C12M 1/34; A61K 39/245; A61K 39/21

(52) U.S. Cl. CPC C12N 7/00; C12M 41/12; A61K 39/245; A61K 39/21; C12N 2710/16634; C12N 2710/16651; C12N 2740/16034; C12N 2740/16051; C12N 2770/20034

(57) ABSTRACT

Methods and systems and architecture for producing cells, virus and bacteria of different sizes and structures by parameters control of temperature or humidity. While these parameters can be constant or time dependent. Said systems may comprising heating elements such as electric heaters, incubating chambers and cooling elements.

### BACKGROUND OF THE INVENTION

Viruses, bacteria and cells may change their molecular spatial structure or size or molecular composition according to temperature or humidity values. The immune response may change according to the size and shape of viruses, bacteria and cells antigens. These were reported in numerus experiments and scientific papers.

Virus changes of structures and size according to temperature are reported for example, in Zhang, X. et al pages 6795-6799, vol. 110, PNAS, (2013). This paper reports that Dengue virus structure has conformational transition when changing the temperature from that present in its mosquito vector of room temperature 28° C. to that of its human host of 37° C.: Artmann, G. M. et al. Eur Biophys J 33, pp. 490-496 (2004). Another paper analyzed data related to dsDNA viruses and different temperatures. The results indicate both dsDNA genome length (bp) and virion volume decreased exponentially with increasing temperature, by about 55-fold as the temperature of occurrence increases from 0 to 40° C. Both relationships were highly significant (p, 0.001), with temperature explaining 47% and 40% of the variation in genome length and volume, respectively. Nifong R L, Gillooly J F, Biol. Lett. 12, (2016).

Bacteria Change their Structures According to Temperature.

There are numerus experiments reported of bacteria size change according to temperature. For example, an experiment shows that Escherichia Coli bacteria size is smaller at higher temperature, F. J. Trueba et al'. Archives of Microbiology pp. 235-240, 131, (1982).

Cells Change their Structures According to Temperature.

There are numerous reports of cell size or spatial structure change according to temperature. For example, an experiment indicated that changes in human cells. Human hemoglobin A (HbA) and hemoglobin S (HbS) exhibited accelerated denaturation between 35 and 39° C. with a midpoint at 37.2±0.6° C. Artmann, G M et al. Eur Biophys J, 33, pp. 490-496 (2004).

Another experiment indicated that Candida utilis yeast has smaller size at higher temperature C. M. Brown and A. H. Rose, journal of biotechnology, pp. 261-272, (1969).

Virus, bacteria and cells change their immune responses according to their size or shape. There are numerus reports of dependence of the immune response according to virus, bacteria or cells' size or shape. For example, an experiment indicated that antigen size is one of the important factors regulating the potency of humoral immune response induced by CTLA4 targeted DNA vaccines. Reducing antigen size increased the immunogenicity of antigen. Jia R, Guo J H Fan M W., 12(1), pages 21-5 Int Immunopharmacology, 2012. Another report states that size and shape of antigen affect the immune responses to said antigen, Bachmann, M et al, Nature Reviews Immunology 10, 787-796(2010). Another report assessed the influence of order on B cell induction and antibody production with the glycoprotein of vesicular stomatitis virus serotype Indiana [VSV-G (IND)]. VSV-G (IND). In VSV-G (IND) transgenic mice, B cells were unresponsive to the poorly organized VSV-G (IND) present as self antigen but responded promptly to the same antigen presented in the highly organized form. Thus, antigen organization influences B cell tolerance. Bachmann MF et al., pp. 1448-1451, Science, (1993).

### SUMMARY

Viruses, bacteria and cells may change their molecular ingredients or their spatial structure or size or molecular structure or any combination of these according to the temperatures they are subjected to.

One objective is to prepare from a group of viruses or cells such as bacteria, having essentially or approximately the same genetic sequence, subgroups of viruses or cells such as bacteria, with different sizes, shapes or genome lengths, or genetic sequence or any combination of these, by dividing said group to initial sub groups and applying on the initial sub-groups different temperatures values. One objective is to produce multiple variants of the same sub group of viruses or bacteria or cells for production of a mixture or set of vaccines, each intended for a single recipient. These variants are created by incubation of initial sub-groups at different temperatures and optionally also different other conditions.

According to one aspect a method is provided for preparing viruses or bacteria or cells, modifications with different structure or sizes or multiplications of genetic sequences or mutations by applying on them different temperature values for supplying them for pharmaceutical or medical research and/or development.

According to another aspect, a method is provided for preparing from an initial group of the same alive viruses, or cells such as bacteria, sub-groups of different alive variants, with the sub-groups characterized by the members having different sizes, mutations, shapes and/or multiplication of genetic sequences between the sub-groups, by dividing said initial group to subgroups and applying to the sub-groups of said same live viruses, cells or bacteria different temperature values. The different types of viruses or cells such as bacteria may be provided for biological or biotechnological applications. For example, but not limited to, development of antibiotics, probiotics, starter cultures, insecticides, enzymes, fuels, hormones, nucleic acid solvents or agriculture.

In the present invention mutation defined as an alteration in the nucleotide sequence of the genome, either DNA or RNA, of virus or bacteria or cell. In the present invention variant defined as virus or bacteria or cell whose genome sequence differs from that of a reference virus or bacteria or cell respectively.

In present invention strain defined as virus or bacteria or cell variant that possesses unique and stable phenotypic characteristics. It may referrer to the original species of virus or bacteria or cell as well.

In the present invention the definition of pathogen may include human or animals cells for example cancer cells or autoimmune disease cells.

The described methods can begin with subgroups comprising the same alive viruses or bacteria or cell without preparing first a single initial group for distribution.

Another objective is to prepare vaccines for viral or bacterial or cell diseases caused by other pathogens, for effective vaccines at different temperature values.

Said live pathogen's temperatures in the vaccine preparation process may be similar to the temperatures the pathogen is subjected to outside the body or to the pathogen temperature inside the body. The vaccine for use can include a mixture of said vaccines that were prepared at different temperature values when said pathogen was live in each preparation or it can be a set of several vaccines each prepared at different temperatures values when said pathogen was alive and taken separately.

The vaccine/s may provide enhanced immunity to said pathogen's mutations or other differences caused by temperature changes inside or outside the body of an intended recipient of the vaccine/s

According to yet another aspect a system is provided for preparation of the enhanced vaccine/s described herein; the system may include a bioreactor, including at least one vessel that culture pathogens and/or host cells.

According to another aspect a fixed temperature or varied temperature in time is set during the production process to be similar to the pathogen temperatures the pathogen may encounter in their natural environment.

### BRIEF DESCRIPTION OF THE DRAWINGS

FIG. 1 is a schematic illustration of a bioreactor.

FIG. 2 is a schematic illustration of a process flow diagram of a continuous tubular bioreactor system.

FIG. 3a is a schematic illustration of three separate vaccine preparation processes.

FIG. 3b is a schematic illustration of a method for preparing a vaccine.

FIG. 4 is a schematic illustration of several separate vaccine manufacturing apparatuses wherein their products are inserted into additional apparatus/es for the following stages in the vaccine preparation.

FIG. 5 is a schematic illustration of a vaccine manufacturing apparatus starting with three separate vaccine processes in three separate apparatuses that continue in a single apparatus.

### DETAILED DESCRIPTION OF THE PRESENT INVENTION

A method is provided of producing from sub-groups consisting of same variant of viruses or bacteria or cells or pathogens, sub-groups with different sizes, shapes, multiplications of genetic sequences or mutations by applying to each sub-group different temperatures in at least one stage of the vaccine preparation.

A method is provided of producing a mixture of vaccines, wherein each vaccine is based on different mutations or other modifications of different sizes or spatial structures of said initial variant or strain. Each vaccine in the mixture is prepared by exposing the pathogens or cells to different temperature conditions in at least one stage of the vaccine preparation process.

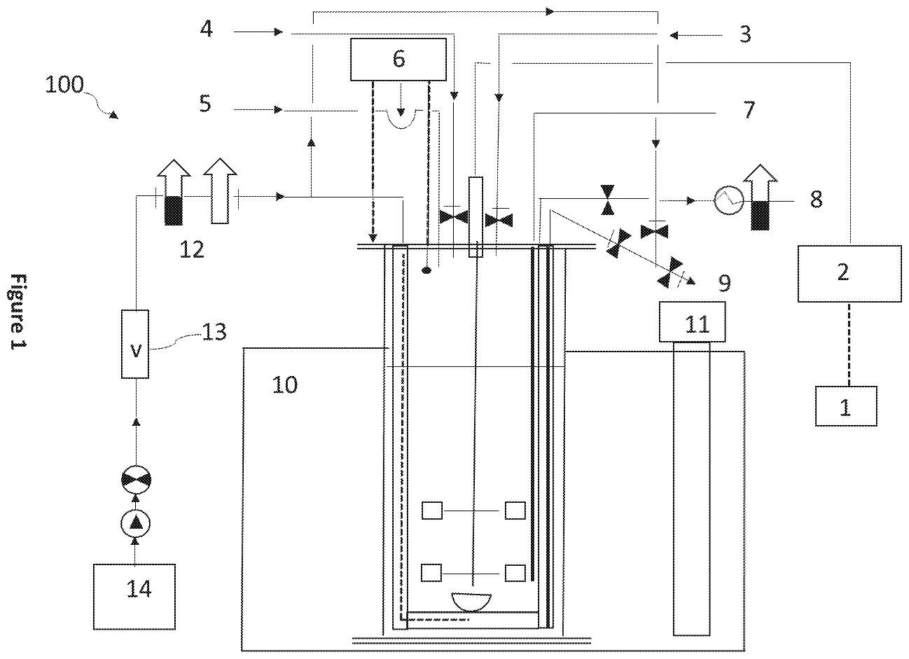

FIG. 1 is a schematic illustration of a bioreactor embodiment for growing viruses or bacteria or cells. Vaccine pathogens in the active state may be stored and/or grown in the bioreactor. Virions can be grown on cells such as chicken embryos or cell lines that repeatedly reproduce. Bacteria can be grown in a bioreactor for bacterial vaccines. In some embodiments some antigens may be manufactured within bacteria or yeast.

With reference to FIG. 1, the bioreactor includes: 1. An engine regulator 2. engine 3. inoculum 4. carbon source 5. anti-foam 6. anti-foam controller 7 thermometer 8. air exhaust system with filter and condenser 9. sampler 10. water bath 11. temperature controller 12. air filters. Said bioreactors may also involve temperature probes, heat transfer system (jacket, coil). Heating may be provided by electric heaters and steam generated in boilers and cooling may be provided by cooling water produced by cooling towers or refrigerants such as ammonia. Said temperature controller 11 can be a chip that sets the temperature value, the period of time of each temperature or a program of temperature value variations over time. The chip may also control the electric heater and flow of the cooling water. Said system may include a humidity regulator for controlling the humidity value inside the containers and bioreactor. These temperatures values regulated by controller 11 are the temperatures values required to produce the different mutations of the pathogens.

Fixed temperatures or varied temperatures in time may be set in this process to be similar to temperatures the pathogen may encounter in specific environments. A pathogen or cell may be produced having a size, molecular structure and/or size structure similar to the pathogens in a specific environment. The pathogen growth stage in the vaccine production process may be done several separate times and may be done with other groups of said initial variant or strain, each with different temperature values to produce different pathogens or variants, each similar in structure to those that said intended recipients may encounter in certain weather conditions, or in certain places or at certain body temperatures.

A following step in the vaccine process may be to extract the vaccine antigens. The vaccine type can be for example inactivated bacteria or virus, and/or attenuated virus or bacteria or other cell, pathogens or antigens for: conjugate vaccines, bacterial vector-based vaccines, m-RNA vaccines, trained immunity-based vaccines, viral vector-based vaccines, recombinant vector vaccines, DNA vaccines, or pathogens for subunit vaccines.

Wherein the antigens types can be for example but not limited to, different proteins, different RNA sequences, different DNA sequences, different protein spikes, different attenuated or inactivated viruses, different proteins spatial structures, pathogen subunits, toxins, polysaccharide, virus like particles, outer Mestraimbran vesicles, DNA plasmid or mRNA.

In cases wherein there is more than one variant of the pathogen from several vaccine preparations, the completed vaccine may be a mixture of these variants. Said mixture can be administered as a single vaccine or as several independent vaccines. As a matter of course, after said vaccine is inserted into the intended recipient's body, the body generates antibodies.

The vaccine generation method makes the immune system of said vaccine recipient generate different antibodies, T-lymphocytes and B-lymphocytes appropriate to vaccinating the body against the pathogen virus or bacteria or other cell, the pathogen having various spatial structures or molecular structures or mutations as they are expressed in different environments, different temperatures values, or at different body temperatures, making the vaccine more robust and more efficient.

If the pathogens invade the immunized body, the body's immune system is ready to respond to the different versions or variants of said virus or bacteria. In this second response the appropriate memory T-lymphocytes formed in said vaccination stage, may detect this pathogen and B-lymphocytes may produce antibodies to attack said viruses or bacteria.

In some vaccine preparation embodiments the pathogen in the active state is exposed to different of temperature regimes, for each of said different preparations.

In some other vaccine preparation embodiments the temperatures of the pathogens or hosts in active or attenuated state have different values at the same time in different regions of the growing or storing system. This can be accomplished for example, by dividing the space inside the bioreactor to capsules, each at a different cultivating temperature, or by creating a temperature gradient inside the bioreactor.

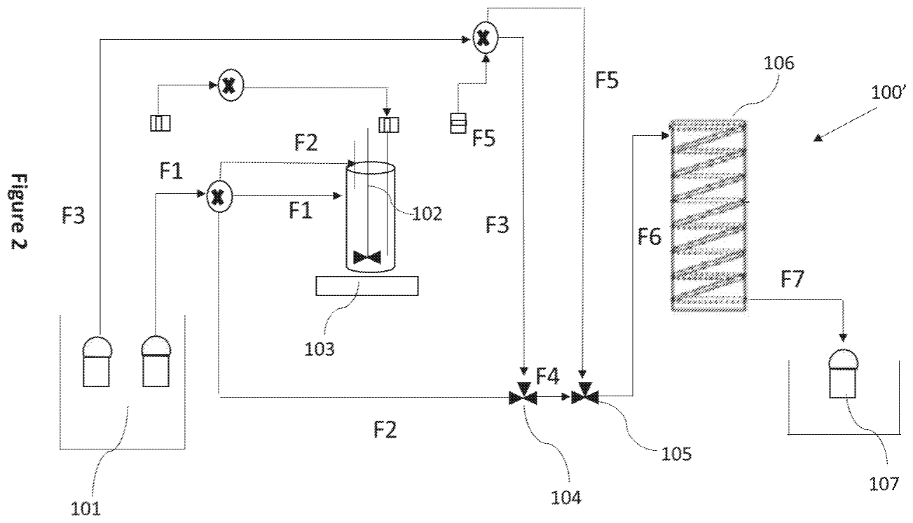

FIG. 2 is a schematic illustration of a process flow diagram of a continuous tubular bioreactor system 100′ which may be suitable for the preparation of the vaccines described herein. For example, the system comprises a bioreactor system for influenza virus production. The system 100′ comprises a Styrofoam box with ice 101 containing a medium stock and a virus stock. The medium stock is fed in stream F1 and host cells and culture medium are in stream F2 and are combined in the 500 mL continuous stirred tank bioreactor (CSTR) 102 which is continuously stirred with a magnetic stirrer 103.

Both cells and culture medium and the virus stock, trypsin and culture medium are streamed F2, F3 respectively to the point of infection 104 where both streams are combined.

The combined streams F4 are then combined with air, with or without CO₂, stream F5, in the air injection port 105. The air injection port 105 produces a new stream of liquid and gas containing cells, virus, trypsin, air and culture medium F6.

The combined stream F6 goes into a 211 mL tubular plug-flow bioreactor (PFBR) 106. The final stream 107, as output from the PFBR, is to a plug-flow bioreactor harvest which contains a combination of liquid and gas containing cells, virus, trypsin, air and culture medium.

The system 100′ was built with a CSTR 102 and a coiled tubular plug-flow bioreactor (PFBR) 106 in series. The complete bioreactor system was installed inside a cultivation room. According to an embodiment the temperature in the bioreactors and the temperature in the cultivation room are set at different values similar to the different temperatures in which the active pathogen may be found in the world.

The CSTR 102 was operated as a chemostat with a dilution rate of approx. 0.9×μmax. The PFBR 106 was constructed using a transparent silicone tube that was coiled around a PLEXIGLAS® XT tube of 20 cm internal diameter and 1 m height.

For vaccines made of attenuated or active vector viruses the bio reactor containing the medium stock can be at room temperature. The process may repeat itself with different temperature values similar to different weather conditions or different body temperatures.

Exemplary flow rates: F1=0.15 mL/min of liquid; F2=0.15 mL/min of liquid; F3=0.05 mL/min of liquid; F4=0.05 mL/min of liquid; F5=0.15 mL/min of gas; F6=0.2 mL/min of liquid and 0.05 mL/min of gas; F7=0.2 mL/min of liquid and 0.05 mL/min of gas.

Example of Parameters that May be Used in a Similar Bioreactor for Producing Viruses.

Bioreactor culture system—a 10 m² bioreactor and an in-line tangential flow filtration module is provided. The bioreactor is inoculated at a target seeding density of 2.0×10⁴ cells/cm². Culture parameters are temperature of 35° C., pH of 7.2, and agitation was set to 250 RPM, with a working volume of 1.6 L. An external heated media recirculation loop is connected to the bioreactor to support the high cell density.

Cell expansion—A Vero cell line is cultured in a serum-free media. Cells are expanded using flat stock and cell factories at a seeding density between 1.0 and 1.5×10⁴ cells/cm². Vessels are cultured at 37° C., 5% CO₂. Cells are passaged until a group of approximately 1.0-2.0×10⁹ total viable cells is achieved. At harvest, cell monolayers are washed with 1×Dulbecco's Phosphate-Buffered Saline, to remove excess spent media followed by dissociation with TrypLE CTS Select. Centrifugation is performed to remove TrypLE, and the cells are resuspended in fresh medium Cell counts are performed on a Vi-CELL XR Cell Viability Analyzer.

Cell density and metabolite analysis monitoring—Single-use sampling strips, inserted in the fixed-bed, are removed daily for cell density determination utilizing lysis buffer and nuclei counts. Sampling strips are lysed for 5 min followed by vertexing for 1 min. Nuclei are stained with crystal violet to visualize intact nuclei. Metabolite concentrations are measured daily using the BioProfile® FLEX2 by removing media samples from the aseptic sampling port. A pH offset is performed when offline measures deviate >±0.05 pH units from the online probe reading.

Infection process-Recombinant Vesicular stomatitis virus (rVSV) (minus the glycoprotein G) containing the Lassa virus (LASV) Josiah glycoprotein (VSVΔG/LASVGP)) is provided. The stock VSVΔG/LASVGP, stock titer of 4.9×10⁸ pfu/mL, is used for all infection studies utilizing the bioreactor. Infection of the bioreactor with virus inoculum is performed five days post-seeding or when peak cell density is obtained, evident by nitrogen source depletion as measured by glutamine. Briefly, the bioreactor is drained of spent media and then refilled with fresh media containing the viral inoculum. The recirculation loop is disconnected at the point of infection, to perform a batch mode infection. Each run is infected at a MOI (Multiplicity of Infection) of 0.05, and the infection process proceeds for 48-72 h. Harvest is initiated once cell counts are depleted on the sample strips. Infection of the flatstock vessels occurs at the same time as the bioreactor is refilled, utilizing the same viral infection inoculum.

Viral harvest—Bulk harvest is passed through a two-step depth filtration chain, Sartopure PP3 8 μm followed by a Sartopore 2 0.8/0.45 μm filter and collected into a secondary reservoir. The bulk harvest is concentrated 2-fold using a 100 kDa hallow-fiber TFF cartridge. Flatstock vessels are harvested at the same time as the bioreactor, with the bulk harvest clarified via centrifugation at 1000 g for 10 min. Parameters are from Berrie et al, 3639-3645, Vaccine, 2020.

A method and apparatus of the present invention include a system for preparing a vaccine said system comprise a cultivation container for group of pathogen virus or bacteria or cells of essentially the same variant or strain. In addition, said system comprise an element for heating or cooling or both. Said system comprise a temperature control element to set the temperature in the container and in by controlling the heating and cooling element. The temperature can be constant or varied in time. Said group is divided to several sub-group containing essentially the same variant Wherein each said sub-group is cultivating in different temperature or different temperature variation in time. Result in different mutation or spatial structural change in each sub-group. In another embodiment related to said method and apparatus cultivating said subgroups at different temperatures is realized by using the same system more than one time each time with other subgroup and different temperature parameters or using more than one of said system wherein the subgroup in each system or more is cultivating in different temperatures parameters. In another realization of said method and apparatus said system contains inside more than one unit, each unit cultivating said subgroups at different parameters. Another embodiment of the present invention related to said method and apparatus is the use of said modified subgroups variants to extract antigens for vaccines preparation. Wherein the complete vaccine is a mixture of vaccines prepared with said one or more antigens or a set or one or more vaccines each prepared with one or more of said antigen consuming by the same person. Another embodiment related to said method and apparatus is using said modified variants for biotechnology, pharma and medical applications.

Another embodiment is directed to vaccine manufacturing processes including collecting viruses or bacteria of the same type, for example, SARS-CoV-2 virus, from different places or different weather conditions. The viruses or bacteria or cell or other pathogens of the same type, from different places or different weather, may undergo different vaccine preparation process. The final vaccine is a mixture of the vaccines from said different processes or a set of vaccines of said different processes administrated separately.

Another embodiment comprises a virus inactivating and preserving step including for example use of β-propiolactone, which has the advantages that proteins are not damaged, and the inactivating agent is completely hydrolyzed within hours to non-toxic products.

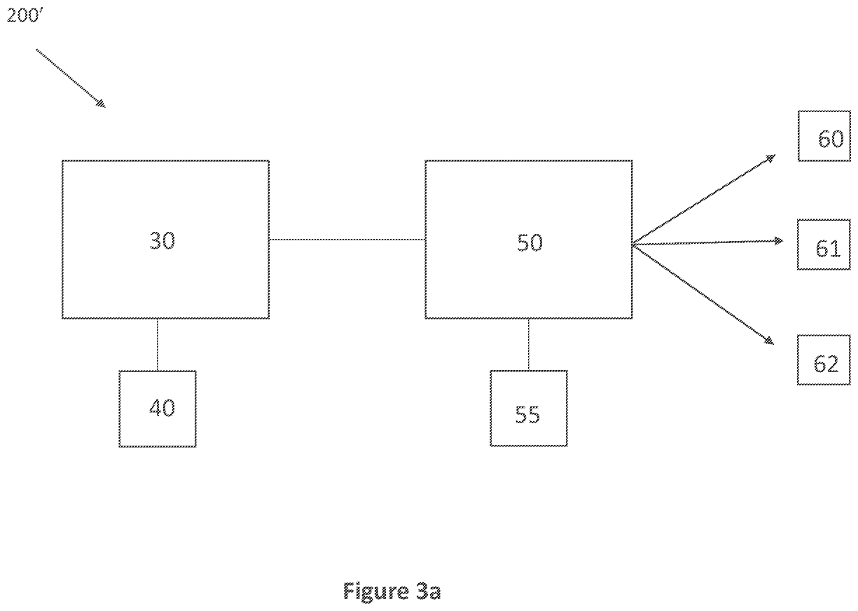

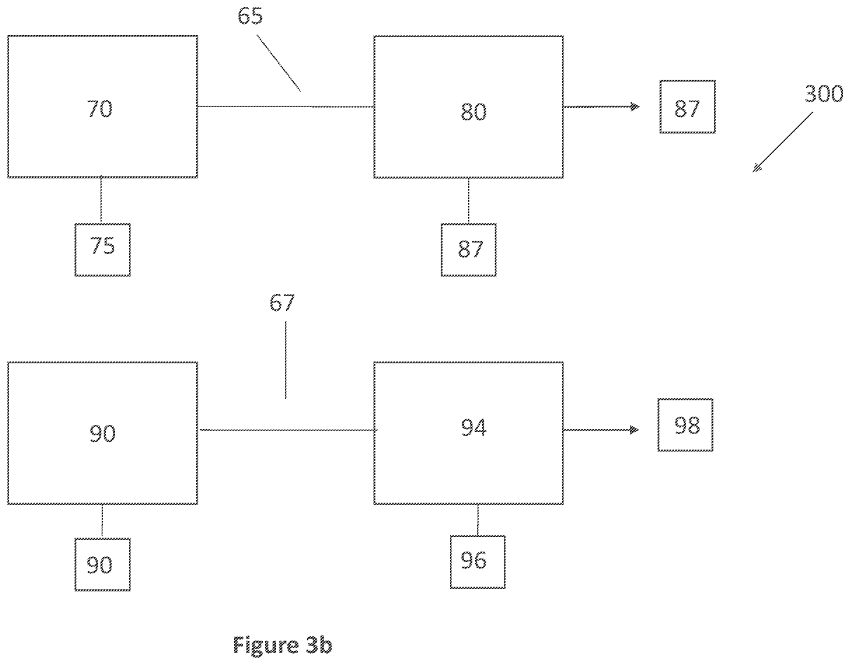

FIG. 3a is a schematic illustration of a system 200′ comprising three separate vaccine preparation processes. The system 200′ comprises a container 30 for the pathogen viruses or bacteria, wherein said viruses or bacteria are stored or incubated. In addition, this system 200′ comprises a climate control system 40 that includes a heating or cooling element or both and may also comprise a humidity controller. The temperature can be constant or varied in time. The controller 40 is used to control temperatures the live viruses or bacteria in container 30 are exposed to.

The method used in the system 200′ may include the stage of inactivating or attenuating the viruses or bacteria and may include the preservation process of said inactivated or attenuated viruses or bacteria. In said method said pathogen temperatures in the live state may be determined according to the possible environments or body temperatures said pathogen could occur at. Block 50 includes all the other stages in vaccine preparation process after incubation. An additional climate control system 55 is associated with block 50 that represents determining the temperature values during the preservation. The vaccine product 60 results from this process. The process 200 described above may be repeated several times, each time with another batch of live viruses or bacteria 30 that is exposed to different temperature values in each cycle, resulting in different virus or bacteria modifications in each cycle and different vaccine products 60, 61 and 62. The final vaccine may contain the products of all three vaccine products 60-62.

FIG. 3b is a schematic illustration of another system 300 for preparing a vaccine. The system 300 comprises several independent systems wherein each exposes the live viruses or bacteria in the production process to different temperature or temperature variations, at least until these viruses or bacteria are inactivated or attenuated.

The usage of different temperatures may produce a number of modifications of the same virus or bacteria or cells variants while using the same starter (primer) modification of the virus. The system 300 includes providing a plurality of independent systems 65, 67 represented for simplicity's sake as two similar systems using different temperatures for producing a vaccine product Each system 65, 67 is associated with a container 70, 90 respectively for the pathogen viruses or bacteria, wherein the live viruses or bacteria are stored or incubated.

Systems 65, 67 comprise a climate control system 75, 92 respectively that includes a heating or cooling element or both and may comprise a humidity control as well. The temperature can be constant or varied in time. Systems 75,92 respectively control the temperature the live viruses or bacteria are exposed to in containers 70, 90 respectively. Operating the system 300 may include a stage of inactivating or attenuating the viruses or bacteria and the system 300 may include the process components for preservation of said inactivated or attenuated viruses or bacteria. In said method and system 300 said pathogen temperature in the live state is determined according to the possible environment or body temperature of said pathogen as it could occur at some place in the world.

For example, a system is provided that contains four separate independent systems similar to the system described above. In the first sub system the culture temperature is 35° C. In the second sub system the culture temperature is 36° C. In the third sub system the culture temperature is 37° C. In the fourth sub system the culture temperature is 37.5° C. Each of the viral harvest reservoirs from said four subsystems is an independent supply for the following stages of vaccine production which can be by any type of vaccine production method. The four final products of vaccines produced may be mixed together and optionally subsequently divided into treatment vaccine doses.

All the following stages in vaccine preparation process are represented in blocks 80, 94 respectively. Additional climate control systems 85, 96 respectively determine the temperature values in preservation components 80, 94 respectively. The vaccine products 87, 98 respectively result from this process. The complete vaccine may contain the products 87, 98 of all said systems 65, 67. Each dose of the vaccine may include some or all of the different products. FIG. 3b is an illustration which is a non-limiting example.

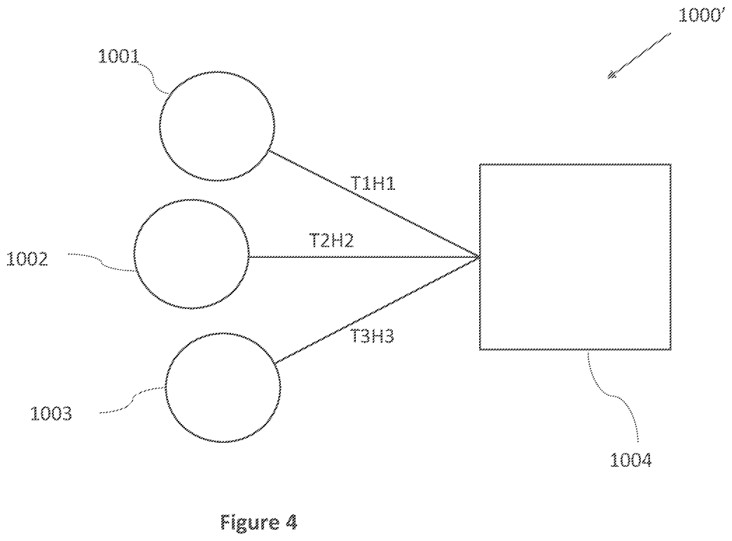

FIG. 4 is a schematic illustration of several separate vaccine manufacturing apparatuses wherein their products are inserted to additional apparatus for the following stages in the vaccine preparation. An apparatus 1000′ for vaccine preparation includes several independent systems for incubating live viruses or bacteria or cells. Each of these separate systems 1001,1002,1003 keep the viruses or bacteria in the active stage at different temperatures T1, T2, T3 respectively and humidity values H1, H2, H3 respectively. These separate systems maintain the temperature and humidity values after the described live viruses or bacteria are inactivated or undergo additional process stages. The combination of all three-virus productions moves to an additional system 1004. The additional system 1004 includes the next stages of the vaccine preparations. Three different modifications that were saved and incubated in the systems 1001, 1002, 1003 are combined in order to produce the final vaccine in a system 1004.

This system 1000′ may be used for creating new modification or mutations from one starter modification by exposing it to different temperatures during the growth and forming of the vaccine products.

The system 1000′ combines the vaccine products into one vaccine, which causes the body to be vaccinated against the virus at different temperatures of the environment or of the body, thereby increasing the efficacy of the vaccine. The presented method and system 1000′ may also cause the body to be vaccinated against future modifications that will be formed due to temperature and season changes.

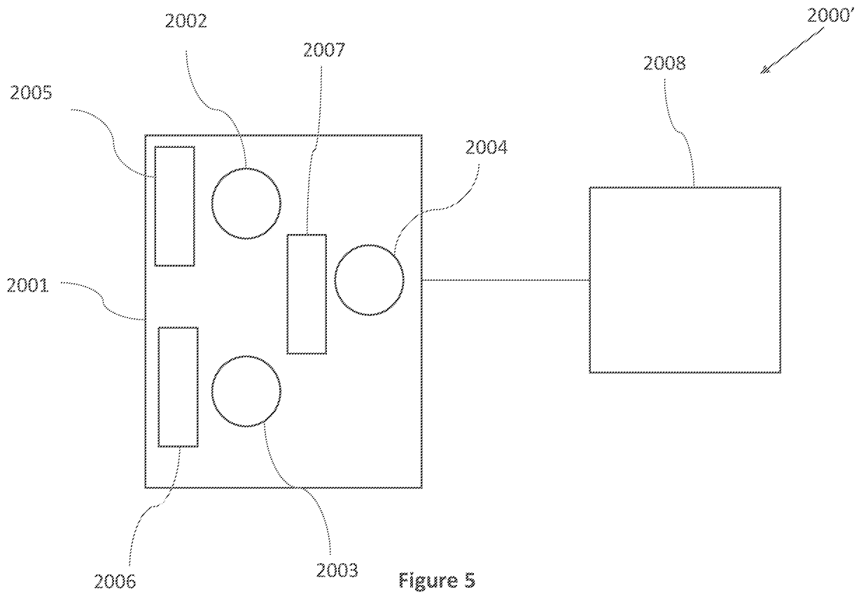

FIG. 5 shows an apparatus 2000′ for vaccine preparation that includes three elements for incubating live vaccines or bacteria or cells 2002, 2003, 2004. Each of the three incubating elements 2002, 2003, 2004 includes climate control elements 2005, 2006, 2007 respectively. The climate control elements 2005, 2006, 2007 keep the viruses or bacteria or cells in the active stage at different temperatures values.

After said live viruses or bacteria are inactivated or undergo additional process stages their products are all moved to a block 280 that is configured to allow performing the next stages of the vaccine preparations.

Another embodiment is a conjugate vaccine/s that comprises one or more parts and/or a DNA vaccine that comprises one or more parts, and/or nanoparticle vaccine/s or non-replicating viral vector vaccines, replication-incompetent vector/s, replication-competent vector/s or inactivated vaccine/s (formalin with alum adjuvant) or protein subunit vaccine/s, that comprise one or more parts, or vector-based m-RNA vaccines that comprise one or more parts, and/or inactivated virus/es that may comprise one or more parts, and/or trained immunity-based vaccine/s that comprise one or more parts. In the preparation of each vaccine part, the pathogen virus or bacteria or cell in the active state is incubated at a different temperature or temperature variation in time or a combination of the above. Said vaccine preparation includes a system for setting the temperature of the pathogen bacteria or viruses in the active state. Said system is set to different temperatures or temperature timelines in each of said partial preparations. Said different temperature values are similar to the pathogen temperatures values at different places or at different times or for different bodies.

In another embodiment related to the apparatuses, systems and method described in the present invention said time varied temperature in the cultivation of virus, bacteria or cell variants is divided to two consecutive temperatures values. Said two temperature values is similar to two different environments or body temperatures.

Another embodiment is an apparatus for delivering the viruses or bacteria from the place they were collected to the vaccine production apparatuses at similar or identical temperature values to those they had at the original collection place. The described apparatus may include a container or storage device for storing the viruses or bacteria. The container may contain a culture for these viruses or bacteria. Adjacent to said container there may be a climate system for keeping said virus or bacteria at the same temperature as at the place from it was taken.

A specific non limiting example of DNA vaccine is in particular for COVID-19 Plasmid DNA. DNA vaccines are based on plasmid DNA that can be produced at large scale in bacteria. Typically, these plasmids contain mammalian expression promoters and the gene that encodes the spike protein, which is expressed in the vaccinated individual upon delivery. The vaccine incorporates several modifications of the spike protein, each taken separately from COVID-19 viruses at different temperature values while they were in the active state and kept at these temperatures values until the viruses are inactivated Said different temperature values may be similar to different weather conditions or different body temperatures. The vaccine may include all of said samples. The great advantage of these technologies is the possibility of large-scale production as well as the high stability of plasmid DNA. However, DNA vaccines often show low immunogenicity, and have to be administered via delivery devices to make them efficient. This requires providing delivery devices, such as electroporators.

Another embodiment refers to replication-incompetent vectors, which are typically based on another virus that has been engineered to express the spike protein and has been disabled from replication in vivo by the deletion of parts of its genome. The vaccine preparation may involve using several modifications of the spike protein, each taken separately from COVID-19 viruses at different temperature values while they were in the active state. These different temperatures are similar to different weather conditions or body temperatures. The vaccine may include all of the samples. The majority of these approaches are based on adenovirus (AdV) vectors. The majority of these vectors are delivered intramuscularly, enter the cells of the vaccinated individual and then express the spike protein, to which the host immune system responds. Another embodiment refers to replication-competent vectors that are typically derived from attenuated or vaccine strains of viruses that have been engineered to express a transgene, in this case the spike protein. In some cases, animal viruses that do not replicate efficiently and cause no disease in humans are used as well. This approach can result in a more robust induction of immunity, because the vector is propagating to some extent in the vaccinated individual and often also triggers a strong innate immune response. Some of these vectors can also be administered through mucosal surfaces, which might trigger mucosal immune responses. In the present embodiment the vaccine uses several modifications of the spike protein, each taken separately from COVID-19 viruses held at different temperature values while they are in the active state. Said different temperatures values are similar to different weather conditions or body temperatures.

A specific non limiting example of recombinant protein vaccine in particular for COVID-19 viruses is described in this embodiment. The recombinant protein vaccine uses a part of the whole protein or a protein fragment thereof such as the RBD or fusion of RBD with a carrier protein as the antigen. In this embodiment said protein or fragment of protein are sampled from COVID-19 viruses that were subject to different temperatures values while in the active state, similar to temperatures in different weather conditions or to different body temperatures. The present vaccine embodiment may include all said processes' products of different samples. Once taken by the antigen-presenting cells (APC), said different antigen proteins may be digested in the endosome, while a small fraction of the digested fragments is trimmed and presented to the major histocompatibility complex (MHC) II molecules, triggering downstream immune responses and thereby generating body immunity against COVID-19 at different weather conditions and body temperatures. The recombinant protein vaccines often require an adjuvant in the formulation to increase the immunogenicity, for example, Matrix-M.

An example of live attenuated vaccines in particular for COVID-19 viruses is described in this embodiment: Live attenuated vaccines are produced by generating a genetically weakened version of the virus that replicates to a limited extent, causing no disease but inducing immune responses that are similar to that induced by natural infection. Attenuation can be achieved by adapting the virus to unfavourable conditions for example, growth in non-human cells or by rational modification of the virus (for example, by codon de-optimization or by deleting genes that are responsible for counteracting innate immune recognition). This process may be repeated separately with COVID-19 viruses at different temperature values while they are in the active state. Said different temperatures values are similar to different weather conditions or body temperatures. The vaccine may include all of said processes products. An important advantage of these vaccines is that they can be given intranasally, after which they induce mucosal immune responses that can protect the upper respiratory tract, the major entry portal of the virus. In addition, because the virus is replicating in the vaccinated individual, the immune response is likely to target both structural and non-structural viral proteins by way of antibodies and cellular immune responses. A specific non limiting example of the present invention is its use in COVID-19 virus Messenger RNA Vaccine produced via chemical. Since antigen expression from mRNA is a transient process, the risk of host DNA integration is negligible. The elimination of using live materials is an advantage from a quality control standpoint and allows quick product switching in manufacturing facilities. This is because different proteins differ only in the sequence of the RNA molecules, which can be easily modified in the solid phase synthesis process. Naturally occurring mRNA molecules have low apparent transfection efficacy. Therefore, lipid nanoparticles (LNPs) are often used to incorporate the mRNA molecules for transfection purposes. In the present invention the vaccine use several modifications of said mRNA molecules each was replicated or taken separately from COVID-19 viruses that were in different temperatures and or humidity values while they were in the active state in the vaccine preparation. Said different temperatures and or humidity values are similar to different weather conditions or body temperatures. A typical LNP formulation consists of an RNA condensing lipid to form a complex with the mRNA molecule, helper lipids to provide the structural rigidity, and lipidized polymer coating to modify the surface properties of the particles. Once phagocytosed by a cell, the LNPs are exposed to a low pH environment in the endosome, and the RNA condensing lipid can puncture the endosome and allow the mRNA molecule to be released in the cytosol. Therefore, the RNA condensing lipid is the key component of this platform. The 1,2-dioleoyl-3-trimethylammonium-propane (DOTAP) and dilinoleylmethyl-4-dimethylaminobutyrate (DLin-MC3-DMA) are the two common commercially available positively charged lipids for this purpose of COVID-19. LNP-encapsulated mRNA vaccine encodes the S protein, given as two doses by intramuscular (IM) injection. In the vaccine of the present invention several variation of the S protein are encoded each is similar to S protein of live COVID-19 virus at different weather conditions or blood temperature.

Another embodiment of the present invention is its use in viral vector-based vaccines, where the antigen is cloned into a viral vector that lacks the ability to reproduce. Common vectors include lentivirus, adenovirus, and adeno-associated virus (AAV). The viral vector imitates viral infection disease state and therefore can produce stronger cellular immune responses. In the present invention this process repeated on antigens in different temperatures and or humidity values while they are in the active state. Said different temperatures and or humidity values are similar to different weather conditions or body temperatures. For example, said antigen is COVID-19. The vaccine includes all said processes products.

Another embodiment of the present invention is its use in COVID-19 bacterial vector-based vaccines. Such as, the non-pathogenic lactic acid bacteria. Where the antigen is from COVID-19. In the present invention this process repeated on antigens in different temperatures and or humidity values while in they are in the active state. Said different temperatures and or humidity values are similar to different weather conditions or body temperatures. For example, said antigen is COVID-19. The vaccine includes all said processes products.

Another embodiment involves use of COVID-19 inactivated vaccines. Such vaccines may be produced by growing COVID-19 in cell culture, usually on Vero cells. In the present embodiment this process is repeated separately on COVID-19 viruses at different temperatures values while they are in the active state. Said different temperatures values are similar to different weather conditions or body temperatures at which said viruses can be found. The incubation is followed by chemical inactivation of the virus. The vaccine may include all of said processes products. They can be produced relatively easily; however, their yield could be limited by the productivity of the virus in cell culture and the requirement for production facilities to be at biosafety level 3. These vaccines are usually administered intramuscularly and can contain alum (aluminum hydroxide) or other adjuvants. Because the whole virus is presented to the immune system, immune responses are likely to target not only the spike protein of COVID-19 but also the matrix, envelope and nucleoprotein.

Another embodiment involves generating the pathogen with different desirable sizes and molecular structures that are similar to the pathogen in virus or bacteria, in active state, in different environments, by exposing the virus or bacteria to one or more of these options: electric field, magnetic field, electromagnetic waves or radiation, ultra sound, chemical substrate and chemical ingredients.

Another embodiment is directed to a method and apparatus for designing COVID-19 detectors. Said detectors comprise means to detect COVID-19. A detector will be trained to detect and adjust to different strains of COVID-19 viruses or parts of COVID-19 viruses that while at active state occur at different temperatures, making said detection apparatus able to detect different structured COVID-19 virions that are similar to the different versions of COVID-19 found at different weathers conditions. This apparatus can be realized for example by said detector being trained on COVID-19 contained in a culture, with the COVID-19 temperature set by an adjacent heating element.

A specific non limiting example is the use of the described method is the use for preparing vaccines for cancer diseases, which are effective in different temperatures values. Making the vaccine users immune to said vaccine antigen at different temperatures values inside the body and the external environment. By preparations of vaccine, where the antigen or pathogen that vaccinates the body against cancer have different temperature at each vaccine preparation process. For the purpose of receiving an effective vaccine against the pathogen or antigen at said temperatures. Where said antigen temperatures in the vaccine preparation process may be similar to the temperature outside the body or the temperature inside the body. The vaccine can consist of a mixture of said vaccines that were prepared at different temperatures values on each preparation or it can be a set of several vaccines each prepared at different temperatures values and taken separately. The antigen changes its confirmation, size or structure according to the temperature values Preparing the vaccines at different temperature values result in a vaccine that contains different antigens that appropriate to different body or environment temperatures which result in, people that are immune to cancer at different body and environment temperatures values, which is not necessary the case at casual vaccine preparation.

An additional option is preparing vaccine/s produced from collected viruses or bacteria from environments with different temperature values and keeping the viruses or bacteria at said values at least till they are inactivated or attenuated in the vaccine production process.

Another specific non limiting example is the use of the described method in Herpes simplex virus HSV Sub unit vaccine preparation process. The HSV virus while in live or active state are exposed to different temperatures in the bioreactor by putting the viruses in different independent units inside the bioreactor, each unit has different temperature at the end of the independent stages their products are gathered together. Additional option is by repeating the vaccine production process several times, each time with other viruses exposed to a different temperature.

An additional option is collecting live HSV viruses from different places or seasons with different weather conditions, and placing each sample in a different cycle or a different bioreactor unit in the bioreactor, exposing it to the same temperature as it was collected. An additional option is that said viruses are kept at said temperature conditions in the vaccine process until said viruses are inactivated and preserved by a preservation method that keeps them in the same structure regardless of the change in their temperature. The subunit HSV vaccines are mainly glycoprotein mixes purified on lectins (e.g., lentil lectin) showing high affinity to the HSV virion envelope antigens. The dominant protective antigens among the 11 HSV glycoproteins are gB and gD, which possess important immunogenic epitopes. The abovementioned glycoproteins elicit virus-neutralizing antibodies, antibodies participating in the antibody-dependent cellular cytotoxic (ADCC) response, and they also activate the T lymphocytes. Several reports described the efficacy of the subunit vaccines of various purity based on either HSV-1 and/or HSV-2 envelope glycoproteins. For example, the subunit ISV-1 vaccine (strain HSZP Immuno) had been prepared from chick embryo cells infected with the low-virulent HSZP strain tested in cooperation with the Research and Development Department of the former Immuno AG Company in Vienna. The infected cell extract may be purified on lentil lectin to obtain a glycoprotein mix containing at least four envelope glycoproteins (gB, gC, gD and gG). This subunit vaccine may be immunogenic and protective in mice as well as rabbits and show at least partial cross protection in the HSV-2-challenged guinea pigs infected by the vaginal route. All products of the process preparation cycles are gathered to the complete vaccine.

A specific non limiting example is the use of the described method is for producing vaccines for animals. A specific non limiting example is for farm animals, a Salmonella virus vaccine preparation process. The methods and systems described above may be adapted for such preparations Salmonella viruses may be grown separately on Salmonella shigella agar for 24 hr at a different temperature for each vaccine preparation cycle. Said preparation cycle temperatures are temperatures the Salmonella virus is found at in different places or at different seasons or similar to different body temperatures of farm animals. Then separate colonies may be inoculated in tryptase soya broth in a gradual quantity and incubated for 24 hr at the same temperature. Bacteria may be concentrated by centrifugation and the separate final suspension from each prepared and the count adjusted 10e CFU/final dose. Inactivation may be performed under stimming with formaldehyde solution 37% o in a 0.2o/o of final concentration. The inactivated cultures can be neutralized with sodium metabisulfite. The inactivated Salmonella strains are gently and thoroughly mixed. This watery phase of the vaccine is then emulsified in an oily phase (Mineral oil adjuvant (Extra white oil)+span 80). Thiomersal is added as a preservative in a concentration of 0.05 mg/liter. All products of the process preparation cycles are gathered to the complete vaccine.

It is understood that anything referred to herein in single form applies in plural as well. The virus active or live state refer to the virus state where the virus can replicate or reproduce. It is understood that anything referred herein as body temperature may be any temperature in the range between 32-42 Celsius.

It is understood that anything referred to herein as weather or environment temperature may be any temperature in the range between (−)20 and 56 Celsius.

Another embodiment comprises a container with a temperature a regulating system for producing from the same viruses, bacteria, cells or proteins different sizes or mutations of said viruses, bacteria, cells or proteins respectively dependent on the temperature inside the container. For example, for use in biotechnology, chemistry, drugs, antibiotics, probiotics, starter cultures, insecticides, enzymes, fuels, hormones, nucleic acid solvents or agriculture. Additional embodiment is a method for cancer's vaccine preparation or development. The method is a collection of different vaccine's preparations wherein each vaccine preparation the antigen host cell and or the cell that should become immune to the antigen is at different temperature values similar to the possible temperature values inside a human body or external environment. For developing a group of antigens that can immune the body cells at said possible range of temperature values inside the human and or the outside environment the human body is exposed to. Because different temperature cause said cells to have different structures or confirmations thereby required different structure variations if the antigen for optimized immune response.

A specific non limiting example of the use of the described method in vaccines for HIV diseases, which are effective in different temperatures values. Making the vaccine users immune to said vaccine antigen at different temperatures values inside the body and the external environment. By preparations of vaccine, where the antigen or pathogen that vaccinates the body against HIV have different temperature at each vaccine preparation process. For the purpose of receiving an effective vaccine against the pathogen or antigen at said temperatures. Where said antigen temperatures in the vaccine preparation process may be similar to the temperature outside the body or the temperature inside the body. The vaccine can consist of a mixture of said vaccines that were prepared at different temperatures on each preparation or it can be a set of several vaccines each prepared at different temperatures values and taken separately. The said antigen changed its confirmation, size or structure according to the temperature values is at Preparing the vaccines at different temperature values result in a vaccine that contains different antigens that appropriate to different body or environment temperatures which result in, people that are immune to HIV at different body and environment temperatures values, which is not necessary the case at casual vaccine preparation.

A specific non limiting example of the use of described method is for HIV's vaccine preparation or development. The method is a collection of different vaccine's preparations wherein each vaccine preparation the antigen host cell and or the cell that should become immune to the antigen is at different temperature values similar to the possible temperature values inside a human body or external environment. For developing a group of antigens that can immune the body cells at said possible range of temperature values inside the human and or the outside environment the human body is exposed to. Because different temperature cause said cells to have different structures or confirmations thereby required different structure variations if the antigen for optimized immune response.

Another embodiment is a method of producing a vaccine by the methods and apparatuses described in the previous embodiments, where the different pathogens included in the vaccine are based on the different vaccine's virus or bacteria modifications that can appear in a selected country or area only. The purpose of this method is reducing the number of different pathogens in the vaccine. For example, selecting a virus or bacteria modifications that can be found in Mexico at different times of the year.

It is understood that all the embodiments and claims of this invention may be used for example, on the following diseases, viruses and bacteria:
Adverse vaccine reactions in pets, Anthrax vaccines, Brucellosis vaccine, CircoFLEX, Clostridial vaccine, DA2PPC vaccine, Eastern equine encephalitis⋅
Equine influenza, Leishmaniasis vaccine, Rabies vaccine Virus-Serum-Toxin Act
West Nile fever tetanus⋅ pertussis (whooping cough)⋅ poliomyelitis (polio)⋅ measles rubella⋅ Haemophilus influenzae type b infections⋅hepatitis B⋅
influenza⋅ pneumococcal infections⋅ cholera⋅ hepatitis A⋅
meningococcal disease⋅ plague⋅ rabies⋅ bat lyssavirus⋅ yellow fever⋅
Japanese encephalitis⋅ Q fever⋅ tuberculosis⋅ typhoid⋅ varicella-zoster (chickenpox) cancer, Parkinson Alzheimer, pneumonia, copd Asthma⋅ Cholera⋅
Dengue⋅ Diphtheria⋅ Hepatitis A⋅ Hepatitis B⋅ Hepatitis E⋅
Haemophilus influenzae type b (Hib)⋅Human papillomavirus (HPV)⋅Influenza⋅
Japanese encephalitis⋅Malaria⋅ Measles⋅ Meningococcal meningitis⋅
Mumps⋅ Pertussis⋅Pneumococcal disease⋅ Poliomyelitis⋅ Rabies⋅
Rotavirus⋅ Rubella⋅ Tetanus⋅ Tick-borne encephalitis⋅
Tuberculosis⋅ Typhoid⋅ Varicella⋅ Yellow Fever⋅Campylobacter jejuni⋅
Chagas Disease⋅ Chikungunya⋅Dengue⋅ Enterotoxigenic Escherichia coli⋅
Enterovirus 71 (EV71)⋅ Group B Streptococcus GBS)⋅ Herpes Simplex Virus⋅ HIV-1⋅Human Hookworm Disease⋅ Leishmaniasis Disease⋅ Malaria⋅
Nipah Virus⋅ Nontyphoidal Salmonella Disease⋅Norovirus⋅ Paratyphoid fever⋅
Respiratory Syncytial Virus (RSV)⋅ Schistosomiasis Disease⋅Shigella⋅
Staphylococcus aureus⋅ Streptococcus neumoniae⋅ Streptococcus pyrogenes⋅
Tuberculosis⋅Universal Influenza Vaccine HIV⋅ HIV Vaccine Trials Network⋅The US Military's HIV Research Program MHRP)⋅The International AIDS Vaccine Initiative (IAVI) Malaria⋅ Acinetobacter baumannii⋅ Actinomycetoma⋅
Actinomycosis⋅ Acute prostatitis⋅Anaerobic infection⋅Bacillary peliosis⋅
Bacterial pneumonia⋅Bacteroides ureolyticus⋅ Baggio-Yoshinari syndrome⋅
Barcoo fever⋅ Bartonellosis⋅Biliary fever⋅ Bloodstream infections⋅
Botryomycosis⋅ Bovine campylobacteriosis⋅ Brazilian purpuric fever⋅
Brazilian Purpuric Fever⋅Brodie abscess⋅ Brucella suis⋅ Burkholderia cepacia complex⋅ Buruli ulcer⋅ Campylobacteriosis⋅ Capnocytophaga canimorsus⋅
Cariogram⋅ Carrion's disease⋅CC398⋅ Centor criteria⋅ Chlamydia research⋅ Chlamydia suis⋅ Cholera outbreaks and pandemics⋅ Chronic bacterial prostatitis⋅ Chronic recurrent ultifocal osteomyelitis⋅ Combined periodontic-endodontic lesions⋅ Contagious bovine pleuropneumonia⋅ Copper-silver ionization⋅
Digital dermatitis⋅Diphtheria⋅ Diphtheritic stomatitis⋅ Diseases and epidemics of the 19th century⋅ Enteroinvasive Escherichia coli⋅ Epidural abscess⋅
Epiglottitis⋅Erysipelas⋅ European Working Group for Legionella Infections⋅Far East scarlet-like fever⋅ Fitz-Hugh-Curtis syndrome⋅ Foot rot⋅Gardnerella aginalis⋅
Garre's sclerosing osteomyelitis⋅ Gram-negative bacterial infection⋅
Template:Gram-positive actinobacteria diseases⋅ Granuloma inguinale⋅Haemophilus meningitis⋅ Histophilus somni⋅ Human monocytotropic ehrlichiosis⋅ Hundred days' cough⋅ Interdigital dermatitis in cattle⋅
Legionella⋅Lemierre's syndrome⋅ Leprosy⋅ List of clinically important bacteria⋅ List of microbiota species of the lower reproductive tract of women⋅Listeriosis⋅ Lyme disease⋅Meningococcal disease⋅ Methicillin-resistant Staphylococcus aureus⋅ Mycobacterium⋅ Mycobacterium avium-intracellulare infection⋅ Mycoplasma amphoriforme⋅ Mycoplasma hyorhinis⋅ Mycoplasma pneumonia⋅ Mycoplasma synoviae⋅ Nanobacterium⋅ Necrotizing asciitis⋅
Nocardiosis⋅ Noma (disease)⋅ Nontuberculous mycobacteria⋅
Occupational exposure to Lyme disease⋅ Omphalitis of newborn⋅ Orbital ellulitis⋅ Ornithobacterium hominis⋅ Ornithobacterium rhinotracheale⋅
Osteomyelitis⋅Overwhelming post-splenectomy infection⋅ Paget's abscess
Pasteurella natis⋅ Pasteurella canis⋅ Pasteurella dagmatis⋅ Pasteurella langaa⋅
Pasteurella multocida⋅Pasteurella stomatis⋅ Pathogenic bacteria⋅ Pelvic inflammatory disease⋅Peptostreptococcus anaerobius⋅ Peptostreptococcus, asaccharolyticus⋅ Periodontal abscess⋅ Periorbital cellulitis⋅ Peritonsillar abscess⋅
Pneumococcal pneumonia⋅ Porcine intestinal spirochaetosis⋅ Pott disease⋅
Prevotella bivia⋅ Proctitis⋅ Proteus OX19⋅ Pseudomonas infection⋅
Psittacosis⋅Pyaemia⋅ Pyomyositis⋅ Q fever⋅ Relapsing fever⋅
Retrophaiyngeal abscess⋅ Riemerella anatipestifer⋅ Salmonellosis⋅Serratia infection⋅Shigellosis⋅ Southern tick-associated rash illness⋅Staphylococcal scalded skin syndrome⋅ Staphylococcus aureus⋅ Syphilis⋅ Syphilitic aortitis⋅Tetanus⋅
Toxic shock syndrome⋅ Trench fever⋅ Tropical ulcer⋅Tubo-ovarian abscess⋅
Ureaplasma urealyticum infection⋅ Urogenital tuberculosis⋅Vaginal flora in pregnancy Vancomycin-resistant Staphylococcus aureus⋅ Vertebral osteomyelitis⋅
Vibrio tubiashii⋅ Vibrio vulnificus⋅Waterhouse-Friderichsen syndrome⋅
Whooping cough⋅ Widal test⋅ Xanthogranulomatous osteomyelitis⋅ Yersinia pestis⋅ Yersiniosis

It is understood that all the embodiments and claims of this invention may be used for example, on the following animals and non human diseases, viruses and bacteria:

Infectious Bursal Disease, Infectious Bronchitis and Newcastle Disease. dermatitis, sinusitis, otitis externa, pharyngitis, laryngitis and mastitis that may be induced by Gram-positive or Gram-negative bacteria, dermatophytes and yeasts
pyoderma, Adverse vaccine reactions in pets⋅ Anthrax vaccines⋅ Brucellosis vaccine⋅ CircoFLEX⋅ Clostridial vaccine⋅ DA2PPC vaccine⋅influenza⋅
Leishmaniasis⋅ Rabies vaccine⋅ Virus-Serum-Toxin Act⋅ West Nile fever

It is to be understood that the invention is not limited in its application to the details of construction and to the arrangements of the components set forth in the following description or illustrated in the drawings. The invention is capable of other embodiments and of being practiced and carried out in various ways. Many modifications and other embodiments of the inventions set forth herein will come to mind to one skilled in the art to which these inventions pertain having the benefit of the teachings presented in the foregoing descriptions and the associated drawings. Therefore it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

1. A method comprising:
a group of the same strain or variant of pathogens,
distributing and cultivating the pathogens at a plurality of cultures with
each one of said plurality of cultures at a different temperature, at least until pathogens of at least a first culture differ in size and/or genome length and/or mutation and/or spatial structure and/or molecular composition and/or median size from pathogens of a second culture;
harvesting pathogens of said first and second or more than two of said cultures for extracting antigens for at least one vaccine preparation.

2. (canceled)

3. The method of claim 1, wherein the culture comprises host cells.

4. A vaccine as in claim 1, wherein said vaccine is a mixture of the vaccines produced from the modified pathogens antigens and include or not include a vaccine produced from the original pathogen, said vaccines consumed as single doze or as several separate dozes each contain one or more of the said vaccines.

5. The method of claim 1, wherein two or more of said plurality of cultures are cultivated at different humidity values.

6. The one or more vaccines of claim 5, wherein each different temperature and/or humidity is similar to a temperature and/or humidity at different places the viruses or bacteria or cell pathogens can be found.

7. The method of claim 1, wherein during the incubating the temperature is varied in time.

8. The one or more vaccines of claim 1, wherein the different temperatures are similar to possible different body temperatures of an intended recipients of said one or more vaccines.

9. (canceled)

10. The method of claim 1, wherein each cultivation is in a separate preparation apparatus.

11. The method of claim 1, wherein the vaccines contain different antigens that extracted from different variants and/or produced by different vaccine types methods.

12. A method for preparing a vaccine that consists of more than one vaccine wherein the difference between said vaccines of the same type or species of pathogen and or host, are the pathogen and/or host, size or spatial structure or molecular structure or pathogens mutations or any combination of these.

13. A method as in claim 12, for preparing covid-19 vaccines consist of more than one vaccine, wherein the difference between the included vaccines are the initial pathogen each vaccine is based on, wherein said pathogens differ in size or spatial structure or molecular structure or pathogens mutations or any combination of these.

14. (canceled)

15. (canceled)

16. An apparatus for collecting the viruses or bacteria from the habitats; delivering the viruses or bacteria in one or more secure delivery apparatuses to the temperature control apparatuses at similar temperature and/or humidity values from where the viruses or bacteria are collected.

17. A system for vaccine preparation, the system comprising:
a cultivation container for group of pathogens of essentially the same variant or strain, as in claim 1;
a heating and cooling element; and,
a temperature control element directed to control the temperatures of said heating and cooling element and said cultivation container, wherein (i) said temperatures can be constant or varied in time, (ii) said group of pathogens is divided to several sub-groups containing essentially the same variant or strain, and (iii) each of said several sub-groups is being cultivated in a different temperature, resulting different mutations or spatial structure change in at least one of the sub-groups pathogens, such that the modified variants or strains are used for extracting antigens for a vaccine preparation, and (iv) the vaccine is a mixture of vaccines prepared with said antigens, and (v) said vaccine contains set of one or more vaccines each prepared with one or more of said antigens.

18. (canceled)

19. (canceled)

20. (canceled)

21. A system for COVID-19 detection, the system comprising:
a detector for detecting different strains of COVID-19 viruses or parts of COVID-19 viruses;
and,
(i) said different COVID-19 strains are made by COVID-19 viruses contained in one or more cultures, as in claim 1, and (ii) adjacent heating element/s for exposing each COVID-19 viruses culture to different temperature, and (iii) said strains are used for enabling the detector to detect different structured COVID-19 virions that are similar to the different versions of COVID-19 found at different weathers conditions.

22. (canceled)

23. A method and system as in claim 1, wherein after the variant is cultivated in one temperature it is cultivated in second temperature, said second temperature is related to another weather condition or body temperature.

24. A method or apparatus as in claim 1, wherein the vaccine is for the treatment of cancer, wherein the initial cultivated subjects for producing different antigens are similar cancer cells or similar cancer's pathogens.

25. A method or apparatus as in claim 1, wherein the vaccine is for the treatment of HIV, wherein the initial group of HIV pathogens for producing different variants are the same HIV variants or strain.

26. A method or apparatus as in claim 1, wherein the vaccine is for the treatment of Herpes HSV-1 or HSV-2, wherein the initial group of HSV-1 or HSV-2 pathogens for producing different variants are the same HSV-1 or HSV-2 variants or strain.

27. A system for regulating viruses, cells or bacteria, sizes, shapes and/or multiplication of genetic sequences, the system comprising a regulating element for controlling one or a combination of the following means: electric field, magnetic field, electromagnetic waves or radiation, ultra sound, chemical substrate and chemical ingredients, temperature as in claim 1, or humidity; and,
(i) a container wherein said viruses, cells or bacteria are stored or cultivated and regulated by one or more of said means, (ii) said regulated viruses, cells or bacteria are provided for, vaccines or antibiotics or probiotics or starter cultures or insecticides or enzymes or fuels or hormones or nucleic acid solvents or agriculture.

28. (canceled)

29. (canceled)
